(19) United States
(12) Patent Application Publication
(10) Pub. No.: US 2008/0293777 A1
(43) Pub. Date: Nov. 27, 2008

(54) Weight Loss Treatment

(76) Inventors: Daniel A. Erlanson, San Francisco, CA (US); Stig Hansen, Kensington, CA (US)

Correspondence Address: HELLER EHRMAN LLP, 4350 LA JOLLA VILLAGE DRIVE #700, 7TH FLOOR, SAN DIEGO, CA, 92122, US

(21) Appl. No.: 12/126,834

(22) Filed: May 23, 2008

Related U.S. Application Data
(60) Provisional application No. 60/939,778, filed on May 23, 2007.

(51) Int. Cl. A61K 31/41; A61K 31/427; A61P 3/04

(52) U.S. Cl. 514/342; 514/359

(57) ABSTRACT

The present invention relates to the use of 2-phenyl-1,2-benzisoselenazol-3(2H)-one and other selenium-containing compounds for weight loss.

### RELATED APPLICATION

This application claims priority to U.S. Provisional Application No. 60/939,778, filed May 23, 2007, the contents of which are hereby incorporated in its entirety by reference.

### STATEMENT OF GOVERNMENT INTEREST

This invention was made with government support under SBIR Grant Number 1 R⁴³ DK063764. Accordingly, the United States Government may have certain rights in this invention.

### FIELD OF THE INVENTION

The present invention relates to the use of 2-phenyl-1,2-benzisoselenazol-3(2H)-one and other selenium-containing compounds for weight loss.

### BACKGROUND

Obesity has reached epidemic proportions globally, with more than 1 billion adults overweight—at least 300 million of them clinically obese—and is a major contributor to the global burden of chronic disease and disability. Obesity and being overweight pose a major risk for serious diet-related chronic diseases, including type 2 diabetes, cardiovascular disease, hypertension and stroke, and certain forms of cancer. The health consequences range from increased risk of premature death, to serious chronic conditions that reduce the overall quality of life.

### DESCRIPTION OF THE DRAWINGS

FIG. 1 illustrates the relative activity of PTP-1B (Protein tyrosine phosphatase-1B) treated with 0-2 equivalents of Compound 1, with or without DTT (dithiothreitol) added afterwards.

FIG. 2 illustrates the mass spectrum of PTP-1B treated with 1 equivalent of Compound 1 for 15 minutes (PTP-1B: Unmodified protein; +1: PTP-1B with one molecule Compound 1; +2: PTP-1B with two molecules of Compound 1).

FIG. 3 illustrates the weight change in leptin-deficient (ob/ob) mice when administered 25 mg/kg Compound 1; 100 mg/kg Compound 1; 50 mg/kg of metformin hydrochloride; 10 mg/kg rosiglitazone maleate; a combination of 25 mg/kg Compound 1 and 50 mg/kg metformin hydrochloride; and a combination of 100 mg/kg Compound 1 and 50 mg/kg metformin hydrochloride.

FIG. 4 illustrates the weight change in db/db mice (mutation mapped to leptin receptor) when administered 25 mg/kg Compound 1; 100 mg/kg Compound 1; 50 mg/kg of metformin hydrochloride; 10 mg/kg rosiglitazone maleate; a combination of 25 mg/kg Compound 1 and 50 mg/kg metformin hydrochloride; and a combination of 100 mg/kg Compound 1 and 50 mg/kg metformin hydrochloride.

FIG. 5 illustrates the percent weight change from day 1 to day 6 in leptin-deficient mice after treatment with (left to right): 25 mg/kg Compound 1; 100 mg/kg Compound 1; 50 mg/kg metformin; 25 mg/kg Compound 1 and 50 mg/kg metformin; 100 mg/kg Compound 1 and 50 mg/kg metformin; 10 mg/kg rosiglitazone; or vehicle (2% carboxymethyl cellulose (CMC) in water).

FIG. 6 illustrates the percent weight change from day 1 to day 8 in leptin-deficient mice after treatment with (left to right): 25 mg/kg Compound 1; 100 mg/kg Compound 1; 50 mg/kg metformin; 25 mg/kg Compound 1 and 50 mg/kg metformin; 100 mg/kg Compound 1 and 50 mg/kg metformin; 10 mg/kg rosiglitazone; or vehicle (2% carboxymethlyl cellulose (CMC) in water).

### DESCRIPTION OF THE INVENTION

The present invention relates to the use of certain selenium-containing compounds for weight loss.

In one aspect, the invention is directed to a weight loss method, comprising administering to a subject in need of weight loss an effective amount of a compound of Formula (I):

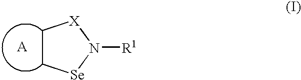

wherein the Se bonded to —N—R¹ above is optionally oxidized;

### X is C═O or SO₂;

A is a 5-6-membered aryl or heteroaryl group, which heteroaryl group has 1 or 2 ring atoms selected from the group consisting of N and S;
in each instance optionally substituted with 1-3 substituents independently selected from the group consisting of straight or branched —C₁₋₆alkyl, straight or branched —C₂₋₆alkenyl, straight or branched —C₂₋₆alkynyl, —OC₁₋₆, —NO₂, —OH, halo, —CN, and —CHO; and

### R¹ is

i) straight or branched —C₁₋₆alkyl, straight or branched —C₁₋₆alkenyl, straight or branched —C₁₋₆alkynyl, —OC₁₋₆, —C₁₋₃—O—C₁₋₃, —OH, —C₁₋₆NR^{a1}R^{b1}, —C₁₋₆(═S)NR^{a1}R^{b1}, —C₁₋₆(═O)NR^{a1}R^{b1}, —NR^{a1}R^{b1}, —CN, —OCN, —C₁₋₆(═O), —C₁₋₆(═O)OH, or —C₁₋₆(═O)OC₁₋₆, or
ii) cycloalkyl, heterocycloalkyl, aryl, heteroaryl, —C₁₋₆alkylcycloalkyl, —C₁₋₆alkylheterocycloalkyl, —C₁₋₆alkylaryl, or —C₁₋₆alkylheteroaryl (wherein for any ring hetero means 1-2 of N, S, O);
in each instance optionally substituted with 1-4 substituents independently selected from straight or branched —C₁₋₆alkyl, straight or branched —C₂₋₆alkenyl, straight or branched —C₂₋₆alkynyl, —OC₁₋₆, —NO₂, —OH, ═O, halo, —CF₃, —NR^{a1}R^{b1}, —CN, —OCN, —C₁₋₆(═O), —C₁₋₆(═O)OH, —C₁₋₆(═O)OC₁₋₆, —OC₁₋₆(═O)NR^{a1}R^{b1}, —C₁₋₆(═O)NR^{a1}R^{b1}, —SC₁₋₆alkyl, —SH, —SO₂NR^{a1}R^{b1}, and —N₃; wherein, in each instance R^{a1} and R^{b1} are independently H or substituted or unsubstituted C₁₋₆alkyl, or one of R^{a1} and R^{b1} is H and the other is C₁₋₆(═O)OH, C₁₋₆C(═O)OC₁₋₆, or —C(═N)N—NO₂, substituted or unsubstituted phenyl or substituted or unsubstituted naphthyl;
   or an isomer or salt thereof.

In one embodiment, the compound of Formula (I) is 2-phenyl-1,2-benzisoselenazol-3(2H)-one. 2-phenyl-1,2-benzisoselenazol-3(2H)-one (CAS No. 60940-34-3) is a well-studied compound in man that has the following structure:

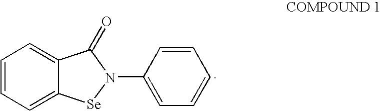

Synthesis of 2-phenyl-1,2-benzisoselenazol-3(2H)-one—also referred to herein as Compound 1—has been described. See, e.g., U.S. Pat. No. 5,008,394; Engman & Hallberg, J. Org. Chem. 54: 2964-2966 (1989); Kamigata et al., Bull. Chem. Soc. Jpn. 59: 2179-2183 (1986).

2-phenyl-1,2-benzisoselenazol-3(2H)-one has been described in the literature to have various biological activities including as a neuroprotective agent; a leukotriene B4 antagonist; an anti-inflammatory agent; prostanoid receptor antagonist; a peroxynitrite scavenger; a glutathione peroxidase mimetic; a peroxide scavenger; and an antioxidant. Shimohashi et al., J. Cell Biochem. 78: 595 (2000); Ramakrishnan et al., Biochem. Pharmacol. 51: 1443 (1996); Ullrich et al., Biochem. Pharmacol. 52: 15 (1996); Dawson et al., Neurosci. Lett. 185: 65 (1995); Wang et al., Hepatology 5: 112 (1992); Maiorino et al., Biochem. Pharmacol. 37: 2267 (1988); Parnham et al., Biochem. Pharmacol. 36: 3095 (1987); Parnham et al., Biochem. Pharmacol. 33: 3247 (1984).

Although 2-phenyl-1,2-benzisoselenazol-3(2H)-one has been the subject of several phase III clinical trials, it has not been approved as a drug by any regulatory authority. The highest level of development for 2-phenyl-1,2-benzisoselenazol-3(2H)-one was when Daiichi filed for regulatory approval in Japan in December of 1997 for cerebral infarction and subarachnoid hemorrhage. However, Daiichi in July 2003 withdrew its application and discontinued development.

The data from the various clinical trials demonstrated that 2-phenyl-1,2-benzisoselenazol-3(2H)-one is cell-permeable, orally bioavailable, and well tolerated in humans. Unlike other selenium-containing compounds, the selenium moiety in 2-phenyl-1,2-benzisoselenazol-3(2H)-one is not bioavailable and this lack of selenium bioavailability is believed to contribute to the low toxicity of 2-phenyl-1,2-benzisoselenazol-3(2H)-one (no toxicity was observed up to 500 mg/day oral dose in humans). Thus, suitable doses in humans range between 1 and 500 mg/day. In another embodiment, a suitable dose is between 100 and 200 mg/day. In another embodiment, a suitable dose is between 150 and 300 mg/day. In another embodiment, a suitable dose is between 1 and 100 mg/kg. In another embodiment, a suitable dose is 10-50 mg/kg. In another embodiment, a suitable dose is between 15 and 30 mg/kg.

In a surprising and unexpected finding, 2-phenyl-1,2-benzisoselenazol-3(2H)-one has been found to be an effective weight loss treatment. In one aspect, the method of the invention comprises administering to a subject in need of weight loss an effective amount of 2-phenyl-1,2-benzisoselenazol-3(2H)-one to induce weight loss in said subject.

In another aspect, the invention is directed to a weight loss method comprising administering to a subject in need of weight loss an effective amount of a compound of Formula (II):

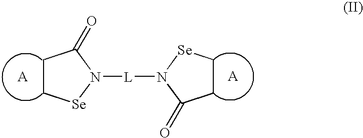

wherein each instance of A is independently a 5-6-membered aryl or heteroaryl group, which heteroaryl group has 1 or 2 ring atoms selected from the group consisting of N and S;
in each instance optionally substituted with 1-3 substituents independently selected from the group consisting of straight or branched —C₁₋₆alkyl, straight or branched —C₂₋₆alkenyl, straight or branched —C₂₋₆alkynyl, —OC₁₋₆, —NO₂, —OH, halo, —CN, and —CHO; and
   L is phenyl, biphenyl, phenyl-CH₂-phenyl, phenyl-CH₂, CH₂-phenyl-, phenyl-SO₂, SO₂-phenyl, -(phenyl)Se—Se(phenyl)- or is straight or branched —C₁₋₆alkyl-, wherein up to three carbon atoms in the alkyl group may be replaced with NR^{a2}, O or S, wherein R^{a2} is H or is —C₁₋₆alkyl; or an isomer or salt thereof.

In another aspect, the invention is directed to a weight loss method comprising administering to a subject in need of weight loss an effective amount of a compound of Formula (III):

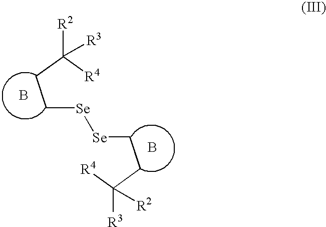

wherein each instance of B is independently an 5-6-membered aryl or heteroaryl group, the heteroaryl group having 1 or 2 heteroatoms selected from the group consisting of N, and S, and wherein the aryl or heteroaryl group may also have a phenyl ring fused thereto and
in each instance B is optionally substituted with 1-3 substituents independently selected from the group consisting of straight or branched —C₁₋₆alkyl, straight or branched —C₂₋₆alkenyl, straight or branched —C₂₋₆alkynyl, —OC₁₋₆, —NO₂, —OH, halo, —CN, and —C₁₋₆(═O)NR^{a3}R^{b3}; wherein in each instance R^{a3} and R^{b3} are independently H or substituted or unsubstituted C₁₋₆alkyl, substituted or unsubstituted —C(═O)C₁₋₆, or substituted or unsubstituted —C(═O)NR^{c3}R^{d3}, wherein one of R^{a3} and R^{b3} is hydrogen, and the other is substituted or unsubstituted phenyl; or wherein R^{a3} and R^{b3} taken together with the nitrogen to which they are attached form a substituted or unsubstituted heterocycloalkyl group;
wherein R^{c3} and R^{d3} are independently H or C₁₋₆alkyl; and
   each instance of R² and R³ is hydrogen or —OH, or one or both instances of R² and R³ attached to the same carbon taken together are ═O; each instance of R⁴ is independently:
i) straight or branched —C₁₋₆alkyl, straight or branched —C₂₋₆alkenyl, straight or branched —C₂₋₆alkynyl, —OC₁₋₆, —C₁₋₃—O—C₁₋₃, —OH, —C₁₋₆NR^{a4}R^{b4}, —C₁₋₆(═S)NR^{a4}R^{b4}, —C₁₋₆(═O)NR^{a4}R^{b4}, —NR^{a4}R^{b4}, —CN, —OCN, —C₁₋₆(═O), —C₁₋₆(═O)OH, or —C₁₋₆(═O)OC₁₋₆, or
ii) cycloalkyl, heterocycloalkyl, aryl, heteroaryl, —C₁₋₆alkylcycloalkyl, —C₁₋₆alkylheterocycloalkyl, —C₁₋₆alkylaryl, or —C₁₋₆alkylheteroaryl (wherein for any ring hetero means 1-2 of N, S, O);
in each instance optionally substituted with 1-4 substituents independently selected from straight or branched —C₁₋₆alkyl, straight or branched —C₂₋₆alkenyl, straight or branched —C₂₋₆alkynyl, —OC₁₋₆, —NO₂, —OH, —O, halo, —CF₃, —NR^{a4}R^{b4}, —CN, —OCN, —C₁₋₆(═O), —C₁₋₆(═O)OH, —C₁₋₆(═O)OC₁₋₆, —OC₁₋₆((═O)NR^{a4}R^{b4}, —C₁₋₆(═O)NR^{a4}R^{b4}, —C(═O)NHC(O)C₁₋₆alkyl, —C(O)NHC(═O)NR^{a4}R^{b4}, —SC₁₋₆alkyl, —SH, —SO₂NR^{a4}R^{b4}, and —N₃;
   wherein, in each instance R^{a4} and R^{b4} are independently H or C₁₋₆alkyl, substituted or unsubstituted, or one of R^{a4} and R^{b4} is H and the other is substituted or unsubstituted cycloalkyl, heterocycloalkyl, aryl, heteroaryl, alkylheteroaryl, —C(═O)heterocycloalkyl, —C₁₋₆(═O)OH, —C₁₋₆C(═O)OC₁₋₆, or —C(═N)N—NO₂;
   or an isomer or salt thereof.

In another aspect, the invention is directed to a weight loss method comprising administering to a subject in need of weight loss an effective amount of a compound of Formula (IV):

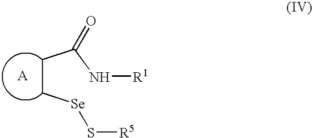

wherein
A is a 5-6-membered aryl, or heteroaryl group, which heteroaryl group has 1 or 2 ring atoms selected from the group consisting of N and S;
in each instance optionally substituted with 1-3 substituents independently selected from the group consisting of straight or branched —C₁₋₆alkyl, straight or branched —C₂₋₆alkenyl, straight or branched —C₂₋₆alkynyl, —OC₁₋₆, —NO₂, —OH, halo, —CN, and —CHO;

### R¹ is

i) straight or branched —C₁₋₆alkyl, straight or branched —C₁₋₆alkenyl, straight or branched —C₁₋₆alkynyl, —OC₁₋₆, —C₁₋₃—O—C₁₋₃, —OH, —C₁₋₆NR^{a1}R^{b1}, —C₁₋₆(═S)NR^{a1}R^{b1}, —C₁₋₆(═O)NR^{a1}R^{b1}, —NR^{a1}R^{b1}, —CN, —OCN, —C₁₋₆(═O), —C₁₋₆(═O)OH, or —C₁₋₆(═O)OC₁₋₆, or
ii) cycloalkyl, heterocycloalkyl, aryl, heteroaryl, —C₁₋₆alkylcycloalkyl, —C₁₋₆alkylheterocycloalkyl, —C₁₋₆alkylaryl, or —C₁₋₆alkylheteroaryl (wherein for any ring hetero means 1-2 of N, S, O);
in each instance optionally substituted with 1-4 substituents independently selected from straight or branched —C₁₋₆alkyl, straight or branched —C₂₋₆alkenyl, straight or branched —C₂₋₆alkynyl, —OC₁₋₆, —NO₂, —OH, ═O, halo, —CF₃, —NR^{a1}R^{b1}, —CN, —OCN, —C₁₋₆(═O), —C₁₋₆(═O)OH, —C₁₋₆(═O)OC₁₋₆, —OC₁₋₆(═O)NR^{a1}R^{b1}, —C₁₋₆(═O)NR^{a1}R^{b1}, —SC₁₋₆alkyl, —SH, —SO₂NR^{a1}R^{b1}, and —N₃; wherein, in each instance R^{a1} and R^{b1} are independently H or substituted or unsubstituted C₁₋₆alkyl, or one of R^{a1} and R^{b1} is H and the other is C₁₋₆(═O)OH, C₁₋₆C(═O)OC₁₋₆, or —C(═N)N—NO₂, substituted or unsubstituted phenyl or substituted or unsubstituted naphthyl; and

### R⁵ is:

i) straight or branched —C₁₋₆alkyl, straight or branched —C₂₋₆alkenyl, straight or branched —C₂₋₆alkynyl, —C₁₋₃—O—C₁₋₃, —C₁₋₆NR^{a5}R^{b5}, —C₁₋₆(═O)NR^{a5}R^{b5}, —NR^{a5}R^{b5}, —C₁₋₆(═O), —C₁₋₆(═O)OH, or —C₁₋₆(═O)OC₁₋₆, or
ii) cycloalkyl, heterocycloalkyl, aryl, heteroaryl, —C₁₋₆alkylcycloalkyl, —C₁₋₆alkylheterocycloalkyl, —C₁₋₆alkylaryl, or —C₁₋₆alkylheteroaryl (wherein for any ring hetero means 1 of N, O);
in each instance optionally substituted with 1-2 substituents independently selected from straight or branched —C₁₋₆alkyl, halo, —C₁₋₆(═O)OH, and —C₁₋₆(═O)OC₁₋₆;
   wherein, in each instance R^{a5} and R^{b5} are independently H or substituted or unsubstituted C₁₋₆alkyl, or —NR^{a5}R^{b5} is —NH(phenyl), which phenyl is substituted or unsubstituted; or an isomer or salt thereof.

In another aspect, the invention is directed to a weight loss method comprising administering to a subject in need of weight loss an effective amount of a compound of Formula (V):

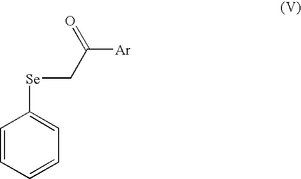

wherein Ar is an aryl or heteroaryl group, which heteroaryl group has 1 ring atom selected from the group consisting of N, S, and O;
in each instance optionally substituted with 1-2 substituents independently selected from the group consisting of straight or branched —C₁₋₆alkyl, —OC₁₋₆, halo, —CN, —OCN, —C₁₋₆(═O), —C₁₋₆(═O)OH, —C₁₋₆(═O)OC₁₋₆, and —OC₁₋₆(═O); or an isomer or salt thereof.

In another aspect, the invention is directed to a weight loss method comprising administering to a subject in need of weight loss an effective amount of a compound of Formula (VI):

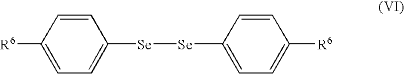

wherein each R⁶ is independently selected from the group consisting of straight or branched —C₁₋₆alkyl, straight or branched —C₂₋₆alkenyl, halo, —CF₃, —NO₂, —CN, —C₁₋₆NR^{a6}R^{b6}, NR^{a6}R^{b6}, —OH, —OC₁₋₆, —OC₁₋₆(═O) and phenyl,
wherein in each instance R^{a6} and R^{b6} are independently H, substituted or unsubstituted C₁₋₆alkyl; substituted or unsubstituted —C(═O)C₁₋₆, or substituted or unsubstituted —C(═O)NRC^{c6}R^{d6}, or wherein one of R^{a6} and R^{b6} is hydrogen, and the other is substituted or unsubstituted phenyl, or wherein R^{a6} and R^{b6} taken together with the nitrogen to which they are attached form a substituted or unsubstituted heterocycloalkyl group and wherein R^{c6} and R^{d6} are independently H or C₁₋₆alkyl;
   or an isomer or salt thereof.

Unless otherwise defined herein, chemical groups are used in accordance with IUPAC conventions (see Compendium of Chemical Terminology: The Gold Book, Second Edition, A. D. McNaught and A. Wilkinson, Blackwell Science, 1997; www.iupac.org).

The term “stable”, as used herein, refers to compounds that possess stability sufficient to allow manufacture thereof or that maintain their chemical integrity for a sufficient period of time to be detected and or to be useful for the purposes detailed herein.

The term “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties. Thus, as used herein, the term “alkyl” refers to acyclic straight and branched groups derivable from alkanes, and having the formula —CₙH₂ₙ₊₁ by removal of a hydrogen atom. The term “alkenyl” refers to acyclic straight and branched hydrocarbon groups having at least one carbon-carbon double bond. Alkenyl groups include alkadienes, alkatrienes, and the like. The term “alkynyl” refers to acyclic straight and branched hydrocarbon groups having at least one carbon-carbon triple bond. Alkynyl groups include alkadiynes, alkatriynes, and the like.

In certain embodiments, the alkyl, alkenyl, and alkynyl groups employed in the compounds described herein contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative alkyl groups include, but are not limited to, methyl, ethyl, n-propyl, isopropyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, allyl, butenyl, 1-methyl-2-buten-1-yl, and the like. Representative alkynyl groups include, but are not limited to, ethynyl, 2-propynyl (propargyl), 1-propynyl, and the like.

The term “alicyclic”, as used herein, refers to compounds which combine the properties of aliphatic and cyclic compounds and include but are not limited to cyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups include, but are not limited to, cyclopropyl, —CH₂-cyclopropyl, cyclobutyl, —CH₂-cyclobutyl, cyclopentyl, —CH₂-cyclopentyl-n, cyclohexyl, —CH₂-cyclohexyl, cyclohexenylethyl, cyclohexanylethyl, norborbyl moieties, and the like, which may bear one or more substituents.

The term “cycloalkyl”, as used herein, refers specifically to cyclic alkyl groups having three to seven, preferably three to ten carbon atoms. Suitable cycloalkyl groups include, but are not limited to, cyclopropyl, cyclobutyl, cyclopentyl, cyclohexyl, cycloheptyl, and the like, which, as in the case of aliphatic, heteroaliphatic or heterocyclic moieties, may optionally be substituted. An analogous convention applies to other generic terms such as “cycloalkenyl”, “cycloalkynyl”, and the like.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, i.e., in place of carbon atoms. Thus, a 1-6 atom heteroaliphatic linker having at least one N atom in the heteroaliphatic main chain, as used herein, refers to a C₁₋₆aliphatic chain wherein at least one carbon atom is replaced with a nitrogen atom, and wherein any one or more of the remaining 5 carbon atoms may be replaced by an oxygen, sulfur, nitrogen, phosphorus, or silicon atom. As used herein, a 1-atom heteroaliphatic linker having at least one N atom in the heteroaliphatic main chain refers to —NH— or —NR— where R is aliphatic, heteroaliphatic, acyl, aromatic, heteroaromatic, or nitrogen-protecting group. Heteroaliphatic moieties may be branched or unbranched. In certain embodiments, heteroaliphatic moieties are substituted by independent replacement of one or more of the hydrogen atoms therein with one or more moieties including any of the substituents described above.

The term “heterocycloalkyl” as used herein, refers to compounds which combine the properties of heteroaliphatic and cyclic compounds and include but are not limited to saturated and unsaturated mono- or polycyclic heterocycles such as morpholino, pyrrolidinyl, furanyl, thiofuranyl, pyrrolyl, etc., which are optionally substituted with one or more functional groups, as defined herein. In certain embodiments, the term “heterocycloalkyl” refers to a non-aromatic 5-, 6-, or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 2 double bonds and each 6-membered ring has 0 to 2 double bonds, (ii) the nitrogen and sulfur heteroatoms may optionally be oxidized, (iii) the nitrogen heteroatom may optionally be quaternized, and (iv) any of the above heterocyclic rings may be fused to an aryl or heteroaryl ring. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

Additionally, it will be appreciated that any of the alicyclic or heteroalicyclic moieties described above and herein may comprise an aryl or heteroaryl moiety fused thereto. Additional examples of generally applicable substituents are illustrated by the specific embodiments shown in the Examples that are described herein.

In general, the term “aromatic moiety”, as used herein, refers to stable substituted or unsubstituted unsaturated mono- or polycyclic hydrocarbon moieties having preferably 3-14 carbon atoms, comprising at least one ring satisfying the Hückel rule for aromaticity. Examples of aromatic moieties include, but are not limited to, phenyl, indanyl, indenyl, naphthyl, phenanthryl, and anthracyl.

In general, the term “heteroaromatic moiety”, as used herein, refers to stable substituted or unsubstituted unsaturated mono-heterocyclic or polyheterocyclic moieties having preferably 3-14 carbon atoms, comprising at least one ring satisfying the Hückel rule for aromaticity. Examples of heteroaromatic moieties include, but are not limited to, pyridyl, quinolinyl, dihydroquinolinyl, isoquinolinyl, quinazolinyl, dihydroquinazolyl, and tetrahydroquinazolyl.

It will also be appreciated that aromatic and heteroaromatic moieties, as defined herein, may be attached via an aliphatic (e.g., alkyl) or heteroaliphatic (e.g., heteroalkyl) moiety and thus also include moieties such as -(aliphatic)aromatic, -(heteroaliphatic)aromatic, -(aliphatic)heteroaromatic, -(heteroaliphatic)heteroaromatic, -(alkyl)aromatic, -(heteroalkyl)aromatic, -(alkyl)heteroaromatic, and -(heteroalkyl)heteroaromatic moieties. Thus, as used herein, the phrases “aromatic or heteroaromatic moieties” and “aromatic, heteroaromatic, -(alkyl)aromatic, -(heteroalkyl)aromatic, -(heteroalkyl)heteroaromatic, and -(heteroalkyl)heteroaromatic” are interchangeable. Substituents include, but are not limited to, any of the previously mentioned substituents resulting in the formation of a stable compound.

In general, the term “aryl” refers to aromatic moieties, as described above, excluding those attached via an aliphatic (e.g., alkyl) or heteroaliphatic (e.g., heteroalkyl) moiety. In certain embodiments, “aryl” refers to a mono- or bicyclic carbocyclic ring system having one or two rings satisfying the Huckel rule for aromaticity, including, but not limited to, phenyl, naphthyl, tetrahydronaphthyl, indanyl, indenyl and the like.

Similarly, the term “heteroaryl” refers to heteroaromatic moieties, as described above, excluding those attached via an aliphatic (e.g., alkyl) or heteroaliphatic (e.g., heteroalkyl) moiety. In certain embodiments, the term “heteroaryl”, as used herein, refers to a cyclic unsaturated radical having from about five to about ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

Substituents for aryl and heteroaryl moieties include, but are not limited to, any of the previously mentioned substituents, i.e., the substituents recited for aliphatic moieties, or for other moieties as disclosed herein, resulting in the formation of a stable compound.

The terms “alkoxy” (or “alkyloxy”), and “thioalkyl” as used herein refers to an alkyl group, as previously defined, attached to the parent molecular moiety through an oxygen atom (“alkoxy”) or through a sulfur atom (“thioalkyl”). In certain embodiments, the alkyl group contains about 1-20 aliphatic carbon atoms. In certain other embodiments, the alkyl group contains about 1-10 aliphatic carbon atoms. In yet other embodiments, the alkyl group contains about 1-8 aliphatic carbon atoms. In still other embodiments, the alkyl group contains about 1-6 aliphatic carbon atoms. In yet other embodiments, the alkyl group contains about 1-4 aliphatic carbon atoms. Examples of alkoxy groups, include but are not limited to, methoxy, ethoxy, propoxy, isopropoxy, n-butoxy, tert-butoxy, neopentoxy, and n-hexoxy. Examples of thioalkyl groups include, but are not limited to, methylthio, ethylthio, propylthio, isopropylthio, n-butylthio, and the like.

The term “amine” refers to a group having the structure —N(R)₂ wherein each occurrence of R is independently hydrogen, or an aliphatic, heteroaliphatic, aromatic, or heteroaromatic moiety, or the R groups, taken together, may form a heterocyclic moiety.

The term “alkylamino” refers to a group having the structure —NHR′ wherein R′ is alkyl. The term “aminoalkyl” refers to a group having the structure NH₂R′—, wherein R′ is alkyl. Examples of alkylamino groups include, but are not limited to, methylamino, ethylamino, iso-propylamino, and the like.

The terms “halo” and “halogen” as used herein refer to an atom selected from fluorine, chlorine, bromine and iodine.

The term “halogenated” denotes a moiety having one, two, or three halogen atoms attached thereto.

The term “haloalkyl” denotes an alkyl group, as defined above, having one, two, or three halogen atoms attached thereto and is exemplified by such groups as chloromethyl, bromoethyl, trifluoromethyl, and the like.

The term “acyloxy”, as used herein, refers to a moiety of structure —OC(O)R^{F}, wherein R^{F} is a substituted or unsubstituted aliphatic, alicyclic, heteroaliphatic, heteroalicyclic, aryl, or heteroaryl moiety.

The term “acyl”, as used herein, refers to a moiety of structure —C(O)R^{F}, wherein R^{F} is a substituted or unsubstituted, aliphatic, alicyclic, heteroaliphatic, heteroalicyclic, aryl, or heteroaryl moiety.

As used herein Cₘ—Cₘ₊ₚ where m and p are positive integers, refers to straight or branched, saturated or unsaturated, cyclic, acyclic, or alicyclic hydrocarbons.

The invention also relates to methods for treating obesity using certain selenium-containing compounds.

In another aspect, the invention is directed to an obesity treatment method, comprising administering to an obese subject an effective amount of a compound of Formula (I) as defined herein to induce weight loss in said subject.

In another aspect, the invention is directed to an obesity treatment method comprising administering to an obese subject an effective amount of Compound 1 to induce weight loss in said subject.

In another aspect, the invention is directed to an obesity treatment method comprising administering to an obese subject an effective amount of a compound of Formula (II) as defined herein to induce weight loss in said subject.

In another aspect, the invention is directed to an obesity treatment method comprising administering to an obese subject an effective amount of a compound of Formula (III) as defined herein to induce weight loss in said subject.

In another aspect, the invention is directed to an obesity treatment method comprising administering to an obese subject an effective amount of a compound of Formula (IV) as defined herein to induce weight loss in said subject.

In another aspect, the invention is directed to an obesity treatment method comprising administering to an obese subject an effective amount of a compound of Formula (V) as defined herein to induce weight loss in said subject.

In another aspect, the invention is directed to an obesity treatment method comprising administering to an obese subject an effective amount of a compound of Formula (VI) as defined herein to induce weight loss in said subject.

As used herein, the term “subject” means a mammal. In certain embodiments, the mammal is a human. In other embodiments the subject is a non-human primate. In other embodiments, the subject is a domesticated mammal, such as companion animal or pet animal, e.g., canine, feline, murine, etc., or a farm animal, e.g., bovine, equine, ovine, caprine, etc.

In certain embodiments of the present application is a weight loss method comprising administering to a subject in need of weight loss an effective amount of 2-phenyl-1,2-benzisoselenazol-3(2H)-one to induce weight loss or to prevent further weight gain in said subject.

As used herein, the terms “a subject in need of weight loss” means a subject that is at least about 10 percent above its ideal weight. In certain embodiments the subject is at risk of further weight gam.

The compounds disclosed herein may also be used in combination with dietary therapy, behavioral therapy, physical therapy, exercise, and weight loss surgery, or a combination of two or more such therapies. In some embodiments, the subject is on a calorie restricted diet. In some embodiments, the subject engages in or is to engage in a physical exercise or physical therapy regimen. In some embodiments, the subject has undergone, or will undergo, weight loss surgery.

Body mass index (“BMI”), also called the Quetelet number or Quetelet index, is currently the most widely accepted calculation of excess body fat for humans. Developed by Adolphe Quetelet, BMI is calculated by dividing the subject's weight by the square of his/her height (BMI=W/h²). In SI units, BMI is typically given as kg/m²; in English units, BMI is typically given as lb/in². For example, a person who weighs 75 kilograms and stands 1.8 meters tall would have a BMI of 75/(1.82)=23.148 and thus would not be in need of weight loss. However, a person who weighs 100 kilograms and stands 1.8 meters tall would have a BMI of 100/(1.8)²=30.864 and therefore would both be in the “obese” range, and thus in need of weight loss.

The methods of the invention may be used to treat humans having a BMI above the recommended body mass index, i.e., at least in the “overweight” range, or at least in the “obese” range. In one embodiment, a human subject is considered in need of weight loss when his or her BMI is 25 or above. In other embodiments, the methods of the invention may be used for the purpose of treating humans having a body mass index of at least about 25, above 25, at least about 30, or above 30.

As used herein, the term “obese” is when a mammal is at least 20 percent above its ideal weight. In another embodiment, a human subject is obese when his or her body mass index (BMI) is about 30 or above. In another embodiment of any of the disclosed methods, the obese subject has a BMI of between about 30 and about 35. Alternately, the obese subject has a BMI of about 35 or higher.

The methods of the invention may be used to treat humans having a body fat percentage above the recommended body fat percentage, i.e., at least in the “overweight” range, or at least in the “obese” range. The body fat percentage will differ between women and men. Specifically, for women, the methods of the invention may be used to treat a female human having a body fat percentage of at least about 25%, above 25%, at least about 32%, or above 32%. For men, the methods of the invention may be used to treat a male human having a body fat percentage of at least about 14%, above 14%, at least about 18%, above 18%, at least about 25%, or above 25%. Body fat percentage may be estimated using any method accepted in the art, including, for example, near infrared interactance, dual energy X-ray absorptiometry, body density measurement, bioelectrical impedance analysis, and the like.

The methods of the invention may be used to treat humans having a waist circumference above the recommended waist circumference. Waist circumference is another widely used measurement to determine abdominal fat content and risk of obesity. An excess of abdominal fat, when out of proportion to total body fat, is considered a predictor of risk factors related to obesity. Men with a waist measurement exceeding 40 inches are considered at risk. Women are at risk with a waist measurement of 35 inches or greater. In one embodiment, the compounds disclosed herein may be used as a weight loss treatment for a male human with a waist circumference exceeding 40 inches. In another embodiment, the compounds disclosed herein may be used as a weight loss treatment for a female human with a waist circumference exceeding 35 inches.

The phrase, “pharmaceutically acceptable derivative”, as used herein, denotes any pharmaceutically acceptable salt, ester, or salt of such ester, of such compound, or any other adduct which, upon administration to a patient, is capable of providing (directly or indirectly) a compound as otherwise described herein, or a metabolite or residue thereof. Certain exemplary pharmaceutical compositions and pharmaceutically acceptable derivatives will be discussed in more detail herein below.

The term “treating”, as used herein generally means that the compounds disclosed herein can be used in humans or animals with at least a tentative diagnosis of disease or condition. In certain embodiments, compounds disclosed herein will delay or slow the progression of the disease or condition thereby giving the individual a longer life span or a better quality of life.

The term “preventing” as used herein means that the compounds disclosed herein are useful when administered to a patient who has not been diagnosed as possibly having the disease or condition at the time of administration, but who would normally be expected to develop the disease or condition or be at increased risk for the disease or condition. The compounds disclosed herein will slow the development of disease symptoms, delay the onset of disease, or prevent the individual from developing the disease at all. Preventing also includes administration of the compounds disclosed herein to those individuals thought to be predisposed to the disease due to familial history, genetic or chromosomal abnormalities, and/or due to the presence of one or more biological markers for the disease.

Compounds may be prepared by crystallization under different conditions and may exist as one or a combination of polymorphs of compounds disclosed herein. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present invention encompasses use of a compound disclosed herein, its derivatives, its tautomeric forms, its stereoisomers, its polymorphs, its pharmaceutically acceptable salts, its pharmaceutically acceptable solvates, and pharmaceutically acceptable compositions containing them.

In another aspect of the present invention, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds disclosed herein can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound disclosed herein that, upon administration to a recipient, is capable of providing, either directly or indirectly, a compound as otherwise described herein, or a metabolite or residue thereof.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference for all that it discloses. Pharmaceutically acceptable salts of the compounds disclosed herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As described above, the pharmaceutically acceptable compositions of the compounds disclosed herein additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds disclosed herein, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; cyclodextrin-type compounds such as Captisol®; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

According to the present invention, the compounds disclosed herein may be assayed in any of the available assays known in the art. For example, the assay may be cellular or non-cellular, in vivo or in vitro, high- or low-throughput format, etc.

In yet another aspect, a method for the treatment or lessening the severity of overweight or obesity and diseases or conditions associated therewith is provided, comprising administering an effective amount of a compound, or a pharmaceutically acceptable composition comprising a compound, to a subject in need thereof. In certain embodiments of the present invention an “effective amount” of the compound or pharmaceutically acceptable composition is that amount effective for treating or lessening the severity of overweight or obesity and diseases or conditions associated therewith. The compounds and compositions, according to the method of the present invention, may be administered using any amount and any route of administration effective for treating or lessening the severity of overweight or obesity and diseases or conditions associated therewith. The exact amount required will vary from subject to subject, depending on the species, age, and general condition of the subject, the severity of the infection, the particular agent, its mode of administration, and the like. The compounds disclosed herein are preferably formulated in dosage unit form for ease of administration and uniformity of dosage. The expression “dosage unit form” as used herein refers to a physically discrete unit of agent appropriate for the subject, e.g., a human patient, to be treated. It will be understood, however, that the total daily usage of the compounds and compositions disclosed herein will be decided by the attending physician within the scope of sound medical judgment with respect to the human patient. The specific effective dose level for any particular human patient or other subject will depend upon a variety of factors including the disorder being treated and the severity of the disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed, and like factors well known in the medical or veterinary arts.

The pharmaceutically acceptable compositions of the compounds disclosed herein can be administered to humans and other subjects orally, rectally, parenterally, intracisternally, intravaginally, intraperitoneally, topically (as by powders, ointments, or drops), bucally, as an oral or nasal spray, or the like, depending on the severity of the condition being treated. In certain embodiments, the compounds disclosed herein may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

In order to prolong the effect of a compound disclosed herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds disclosed herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage form-s the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound disclosed herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

It will also be appreciated that the compounds and pharmaceutically acceptable compositions disclosed herein can be employed in combination therapies, that is, the compounds and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”.

In general, any of the methods disclosed herein may further employ administration to the subject of one or more additional therapeutic agents. Additional therapeutic agents may be selected from among appetite suppressants, neurotransmitter reuptake inhibitors, dopaminergic agonists, serotonergic agonists, modulators of GABAergic signaling, anticonvulsants, antidepressants, monoamine oxidase inhibitors, substance P (NK1) receptor antagonists, melanocortin receptor agonists and antagonists, lipase inhibitors, inhibitors of fat absorption, regulators of energy intake or metabolism, cannabinoid receptor modulators, agents for treating addiction, agents for treating metabolic syndrome, agents for treating hyperinsulinemia, agents for treating insulin resistance, agents for treating diabetes, peroxisome proliferator-activated receptor (PPAR) modulators; dipeptidyl peptidase 4 (DPP-4) antagonists, agents for treating cardiovascular disease, agents for treating elevated triglyceride levels, agents for treating low HDL, agents for treating hypercholesterolemia, and agents for treating hypertension.

In some embodiments, additional therapeutic agents may be selected from among amphetamines, benzodiazepines, sulfonyl ureas, meglitinides, thiazolidinediones, biguanides, beta-blockers, ACE inhibitors, diuretics, nitrates, calcium channel blockers, and statins.

In some embodiments, additional therapeutic agents may be selected from among phentermine, sibutramine, lorcaserin, orlistat, cetilistat, rimonabant, taranabant, topiramate, gabapentin, valproate, vigabatrin, bupropion, tiagabine, sertraline, fluoxetine, trazodone, zonisamide, methylphenidate, varenicline, naltrexone, diethylpropion, phendimetrazine, repaglinide, nateglinide, glimepiride, metformin, pioglitazone, rosiglitazone, and sitagliptin.

The amount of additional therapeutic agent present in the compositions disclosed herein will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds disclosed herein or pharmaceutically acceptable compositions thereof may also be incorporated into compositions for coating implantable medical devices, such as prostheses, artificial valves, vascular grafts, stents and catheters. Accordingly, the present invention, in another aspect, includes use of an implantable device comprising a compound disclosed herein as described generally above, and in classes and subclasses herein, and a carrier suitable for coating said implantable device. In still another aspect, the present invention includes use of an implantable device coated with a composition comprising a compound disclosed herein as described generally above, and in classes and subclasses herein, and a carrier suitable for coating said implantable device.

Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

In particular, formulations disclosed in U.S. Pat. No. 5,021,242 may be used in the methods of the present invention. In another embodiment, a liposomal dosage form of the compounds described herein can be used in the methods of the present invention. An exemplary liposomal dosage form is described in Tajiri et al., Eur. J. Pharm. Sci. 17: S131-S132, Suppl. 1(2002).

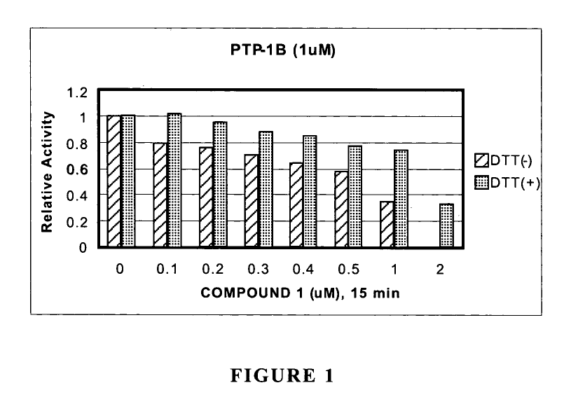

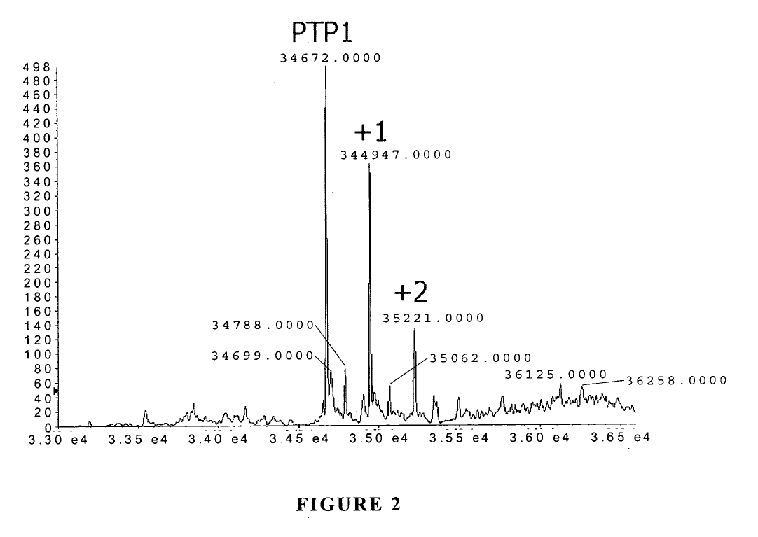

Compound 1's ability to promote weight loss stemmed from another surprising and unexpected finding that Compound 1 is a protein tyrosine phosphatase 1B (PTP-1B) inhibitor. As illustrated by FIGS. 1 and 2, Compound 1 covalently modifies and inhibits the activity of PTP-1B. This covalent inhibition is reversible upon the addition of a reducing agent such as DTT. An endoplasmic reticulum (ER)-associated enzyme expressed in nearly all tissues, PTP-1B plays a major role in modulating metabolism rates and insulin sensitivity through the negative regulation of insulin receptor signaling. Elchebly et al., Science 283:1544-1548 (1999) and Klaman et al., Mol. Cell. Biol. 20: 5479-5489 (2000). PTP-1B also has been reported to be a negative regulator of leptin signaling. Cheng et al., Developmental Cell 2: 497-503 (2002).

To investigate Compound 1's effect on various metabolic disorders including diabetes and obesity, in vivo studies were conducted in the ob/ob obese mouse model and in the db/db diabetic model. The ob/ob mouse is deficient in the hormone leptin, a 16 kDa protein that is secreted by fat cells and plays a key role in regulating energy intake and energy expenditure, including the regulation of appetite and metabolism. Leptin-deficient mice are severely obese and develop diabetes but revert to normal weight upon leptin administration. In the second model, the mice have a genetic mutation mapped to the leptin receptor (db/db) and at the time of the experiment were moderately overweight. Compound 1 was tested in these metabolic disease models along with two anti-diabetic drugs that also affect body weight, metformin hydrochloride and rosiglitazone maleate.

The diet induced obesity (DIO) mouse or rat model is also frequently used in studies of metabolic disorders such as obesity and type 2 diabetes. Typically, mice or rats are fed a high fat diet for 8-12 weeks and become obese and moderately diabetic. The degree of obesity can be controlled by the amount of fat included in the diet

Metformin hydrochloride is an antihyperglycemic agent which improves glucose tolerance in patients with type 2 diabetes, lowering both basal and postprandial plasma glucose. Metformin decreases hepatic glucose production, decreases intestinal absorption of glucose, and increases glucose uptake and utilization. With metformin therapy, insulin secretion remains unchanged while fasting insulin levels and day-long plasma insulin response may actually decrease. Several clinical studies have shown that metformin may reduce body weight in obese patients with or without type 2 diabetes.

Rosiglitazone maleate is a member of the thiazolidinedione class of antidiabetic agents and improves insulin sensitivity while reducing circulating insulin levels. Rosiglitazone is a highly selective and potent agonist for the peroxisome proliferator-activated receptor-gamma (PPARγ).

In humans, PPAR receptors are found in key target tissues for insulin action such as adipose tissue, skeletal muscle, and liver. Activation of PPARγ nuclear receptors regulates the transcription of insulin-responsive genes involved in the control of glucose production, transport, and utilization. In addition, PPARγ responsive genes also participate in the regulation of fatty acid metabolism. Weight gain and increased adipose tissues is one of the significant side-effects of rosiglitazone therapy.

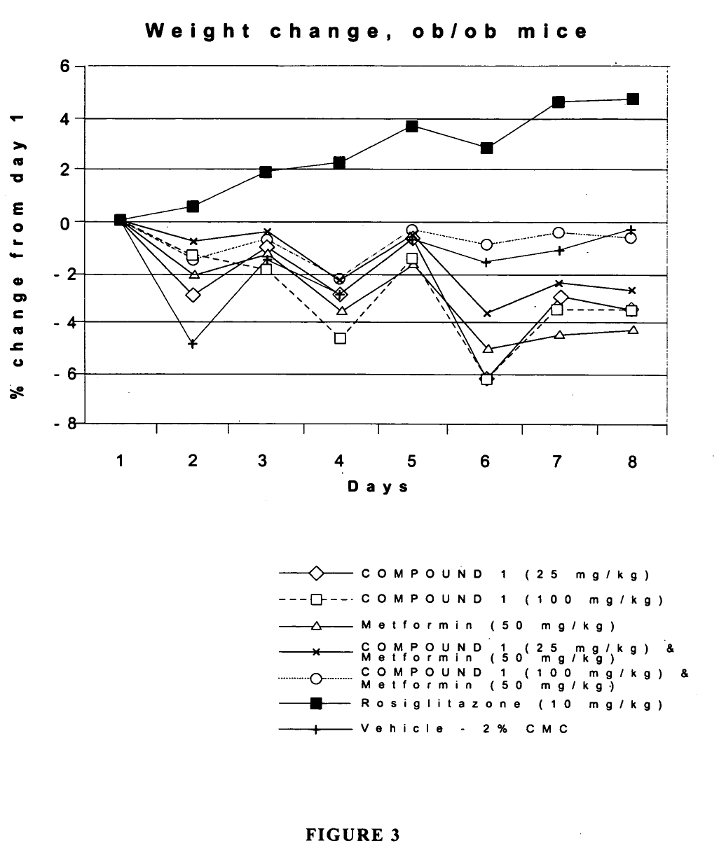

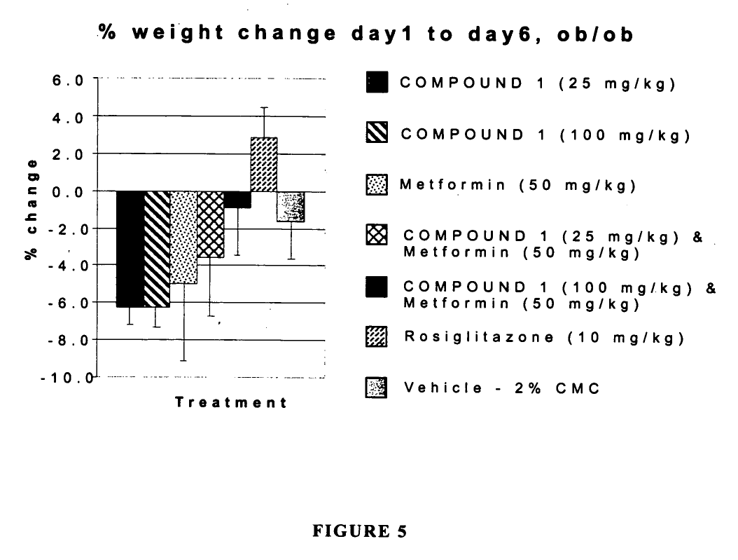

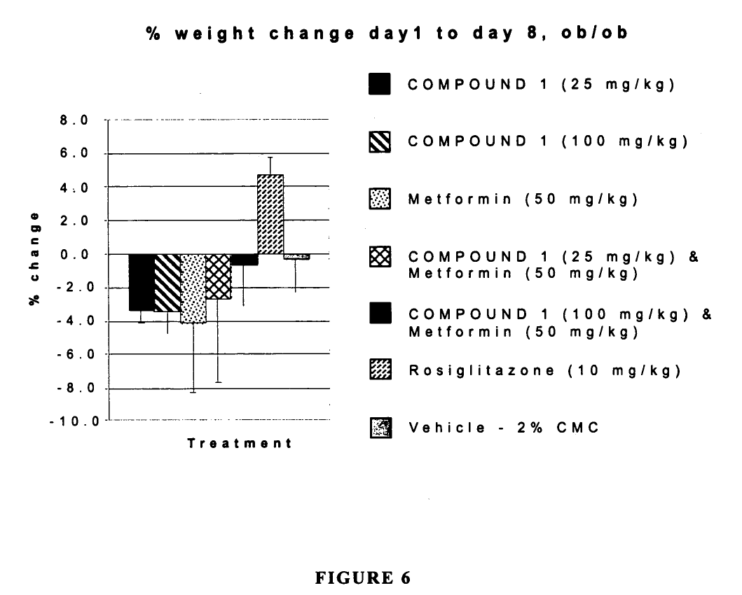

As illustrated in FIG. 3, both Compound 1 and metformin promoted weight loss in leptin-deficient mice. No significant difference in weight loss was observed in mice treated with 20 mg/kg Compound 1 and 100 mg/kg Compound 1 dosed orally BID. Notably, the effects of combining Compound 1 and metformin were not additive and were not more than the effects of either alone. Consistent with clinical experience, rosiglitazone-treated mice gained weight. The observed weight loss in days 6 and 8 with both metformin and Compound 1 were statistically significant in two-tailed T-test with a p-value of less than 0.0005 and 0.005 respective (FIGS. 5 and 6).

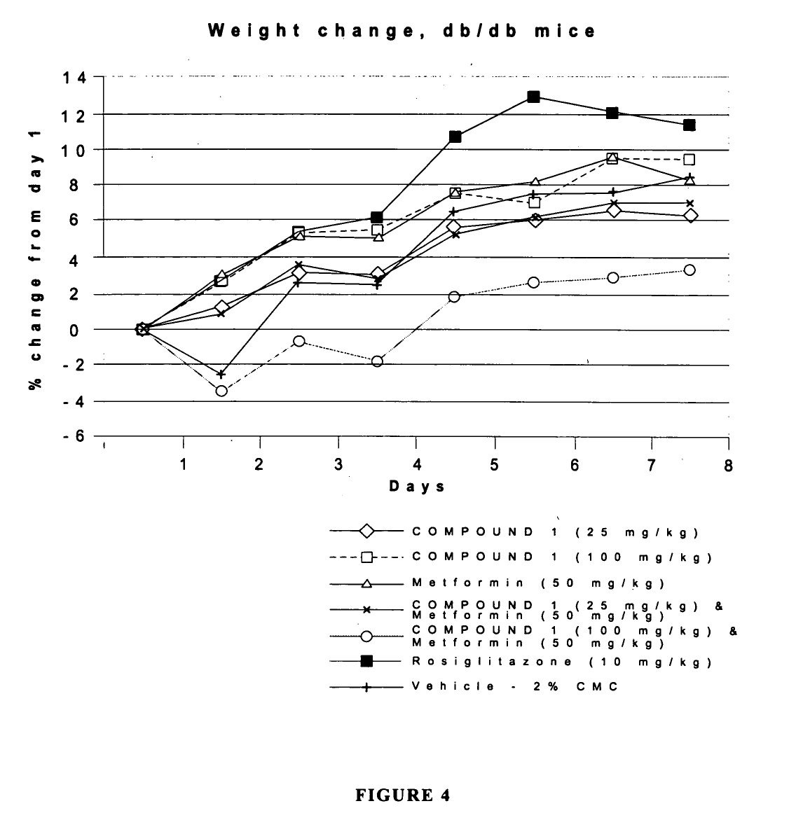

As illustrated in FIG. 4, db/db mice that were only moderately overweight did not lose weight under any regimen and tended to gain weight. However, except for the rosiglitazone-treated mice, the weight gains were not statistically significant.

### Treatment Kit

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the compounds disclosed herein. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### EQUIVALENTS

The representative examples that follow are intended to help illustrate the invention, and are not intended to, nor should they be construed to, limit the scope of the invention. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Example 1

PTP-1B (wild type, residues 1-298) was exchanged into DFTN buffer (DTT-free, 50 mM Tris, 100 mM NaCl, 1 mM EDTA, pH 7.4) using a NAP-5 column, and the concentration of protein was quantified using the absorbance at 280 nm. Purity of the PTP-1B was evidenced by a single mass peak (mass=34,672 amu in a mass spectrometer).

To 99 mL solution of 1.0 μM PTP-1B in buffer was added 1 mL of Compound 1 in DMSO (0 mM, 10 mM, 20 mM, 30 mM, 40 mM, 50 mM, 100 mM, or 200 mM). After 15 minutes, each Compound 1-treated PTP-1B sample was evaluated by mass spectrometry (QSTAR protein mass spectrometer with Baynesian protein mass deconvolution). Concurrently, two 10 μL aliquots were removed. One aliquot was diluted with 10 μL DFTN buffer while the other aliquot was diluted with 10 μL TN buffer (1 mM DTT, 50 mM Tris, 100 mM NaCl, 1 mM EDTA, pH 7.4). After 15 minutes, the samples were diluted with 90 μL pNPP solution (5.6 mM pNPP in DFTN) and the A405 of the solution was monitored for 5 minutes.

### Example 2

Male BKS.Cg-m+/+Leprdb/J (˜5 weeks of age) and B6.V-Lepob/J mice (˜8 weeks of age) were used for in vivo efficacy studies. Mice were housed 4 per cage and acclimated for 10 days prior to the start of the study. Beginning on study day 2, mice were treated PO with the vehicle 2% carboxymethylcellulose, Compound 1 (25 mg/kg and 100 mg/kg), metformin hydrochloride (50 mg/kg), Compound 1 (25 mg/kg and 100 mg/kg) in combination with metformin hydrochloride (50 mg/kg), or rosiglitazone maleate (10 mg/kg), twice per day (BID, morning and afternoon) for 7 days. Mice were weighed each morning just prior to dosing. Mice were fasted overnight between study day 1 and 2 and blood glucose was measured in the morning of day 2 using a glucometer and blood from a tail nick. Food was returned to the mice ad libitum and the mice were again fasted overnight between study day 3 and 4, 5 and 6, and 7 and 8.

### Example 3

The effects of Compound 1 on body weight can also be assessed in long-term studies (3-12 weeks and 4-24 weeks), where ob/ob and/or DIO mice are treated with various doses of Compound 1 daily or intermittently. The inclusion of dosing holidays can be used to assess the duration of the reduced body weight and whether animals start regaining weight. By starting dosing early while the animals are mildly obese, Compound 1 can be evaluated as a weight loss treatment in mildly obese animals and as a method for preventing further weight gain.

What is claimed is:

1. A weight loss method, comprising administering to a subject in need of weight loss an effective amount of 2-phenyl-1,2-benzisoselenazol-3(2H)-one to induce weight loss or to prevent further weight gain in said subject.

2. The method of claim 1, wherein the subject is a human.

3. The method of claim 2, wherein the human is at least about 10 percent above his or her ideal weight.

4. The method of claim 2, wherein the subject is a male human with a waist circumference exceeding 40 inches.

5. The method of claim 2, wherein the subject is a female human with a waist circumference exceeding 35 inches.

6. The method of claim 2, wherein the human has a BMI of about 25 or above.

7. The method of claim 2, wherein the human is at risk of further weight gain.

8. The method of claim 1, wherein the subject is on a calorie restricted diet.

9. The method of claim 1 or 8, wherein the subject is to engage in physical exercise.

10. The method of claim 1, wherein the subject has undergone weight loss surgery.

11. The method of claim 1, further comprising administering to the subject an effective amount of an additional therapeutic agent.

12. The method of claim 11, wherein the additional therapeutic agent is selected from the group consisting of appetite suppressants, neurotransmitter reuptake inhibitors, dopaminergic agonists, serotonergic agonists, modulators of GABAergic signaling, anticonvulsants, antidepressants, monoamine oxidase inhibitors, substance P (NK1) receptor antagonists, melanocortin receptor agonists and antagonists, lipase inhibitors, inhibitors of fat absorption, regulators of energy intake or metabolism, cannabinoid receptor modulators, agents for treating addiction, agents for treating metabolic syndrome, agents for treating hyperinsulinemia, agents for treating insulin resistance, agents for treating diabetes, peroxisome proliferator-activated receptor (PPAR) modulators; dipeptidyl peptidase 4 (DPP-4) antagonists, agents for treating cardiovascular disease, agents for treating elevated triglyceride levels, agents for treating low HDL, agents for treating hypercholesterolemia, and agents for treating hypertension.

13. The method of claim 11, wherein the additional therapeutic agent is selected from the group consisting of amphetamines, benzodiazepines, sulfonyl ureas, meglitinides, thiazolidinediones, biguanides, beta-blockers, ACE inhibitors, diuretics, nitrates, calcium channel blockers, and statins.

14. The method of claim 11, wherein the additional therapeutic agent is selected from the group consisting of phentermine, sibutramine, lorcaserin, orlistat, cetilistat, rimonabant, taranabant, topiramate, gabapentin, valproate, vigabatrin, bupropion, tiagabine, sertraline, fluoxetine, trazodone, zonisamide, methylphenidate, varenicline, naltrexone, diethylpropion, phendimetrazine, repaglinide, nateglinide, glimepiride, metformin, pioglitazone, rosiglitazone, and sitagliptin.

15. An obesity treatment method, comprising administering to an obese subject an effective amount of 2-phenyl-1,2-benzisoselenazol-3(2H)-one to induce weight loss in said subject.

16. The method of claim 15, wherein the subject is human.

17. The method of claim 16, wherein the human is at least about 20 percent above his or her ideal weight.

18. The method of claim 16, wherein the human has a BMI of about 30 or higher.

19. The method of claim 16, wherein the human has a BMI of between about 30 and about 35.

20. The method of claim 16, wherein the human has a BMI of about 35 or higher.

21. A weight loss method, comprising administering to a subject in need of weight loss an effective amount of a compound of Formula (I): 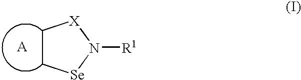
wherein the Se bonded to —N—R¹ above is optionally oxidized;
X is C═O or SO₂;
A is a 5-6-membered aryl, or heteroaryl group, which heteroaryl group has 1 or 2 ring atoms selected from the group consisting of N and S;
in each instance optionally substituted with 1-3 substituents independently selected from the group consisting of straight or branched —C₁₋₆alkyl, straight or branched —C₂₋₆alkenyl, straight or branched —C₂₋₆alkynyl, —OC₁₋₆, —NO₂, —OH, halo, —CN, and —CHO; and
R¹ is
i) straight or branched —C₁₋₆alkyl, straight or branched —C₁₋₆alkenyl, straight or branched —C₁₋₆alkynyl, —OC₁₋₆, —C₁₋₃—O—C₁₋₃, —OH, —C₁₋₆NR^{a1}R^{b1}, —C₁₋₆(═S)NR^{a1}R^{b1}, —C₁₋₆(═O)NR^{a1}R^{b1}, —NR^{a1}R^{b1}, —CN, —OCN, —C₁₋₆(═O), —C₁₋₆(═O)OH, or —C₁₋₆(═O)OC₁₋₆, or
ii) cycloalkyl, heterocycloalkyl, aryl, heteroaryl, —C₁₋₆alkylcycloalkyl, —C₁₋₆alkylheterocycloalkyl, —C₁₋₆alkylaryl, or —C₁₋₆alkylheteroaryl (wherein for any ring hetero means 1-2 of N, S, O);
in each instance optionally substituted with 1-4 substituents independently selected from straight or branched —C₁₋₆alkyl, straight or branched —C₂₋₆alkenyl, straight or branched —C₂₋₆alkynyl, —OC₁₋₆, —NO₂, —OH, ═O, halo, —CF₃, —NR^{a1}R^{b1}, —CN, —OCN, —C₁₋₆(═O), —C₁₋₆(═O)OH, —C₁₋₆(═O)OC₁₋₆, —OC₁₋₆(═O)NR^{a1}R^{b1}, —C₁₋₆(O)NR^{a1}R^{b1}, —SC₁₋₆alkyl, —SH, —SO₂NR^{a1}R^{b1}, and —N₃; wherein, in each instance R^{a1} and R^{b1} are independently H or substituted or unsubstituted C₁₋₆alkyl, or one of R^{a1} and R^{b1} is H and the other is C₁₋₆(═O)OH, C₁₋₆C(═O)OC₁₋₆, or —C(═N)N—NO₂, substituted or unsubstituted phenyl or substituted or unsubstituted naphthyl;
or an isomer or salt thereof.

22. A weight loss method comprising administering to a subject in need of weight loss an effective amount of a compound of Formula (II): 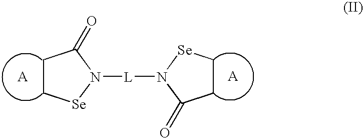
wherein each instance of A is independently
a 5-6-membered aryl, or heteroaryl group, which heteroaryl group has 1 or 2 ring atoms selected from the group consisting of N and S;
in each instance optionally substituted with 1-3 substituents independently selected from the group consisting of straight or branched —C₁₋₆alkyl, straight or branched —C₂₋₆alkenyl, straight or branched —C₂₋₆alkynyl, —OC₁₋₆, —NO₂, —OH, halo, —CN, and —CHO; and
L is phenyl, biphenyl, phenyl-CH₂-phenyl, phenyl-CH₂, CH₂-phenyl-, phenyl-SO₂, SO₂-phenyl, -(phenyl)Se—Se(phenyl)- or is straight or branched —C₁₋₆alkyl-, wherein up to three carbon atoms in the alkyl group may be replaced with NR^{a2}, O or S, wherein R^{a2} is H or is —C₁₋₆alkyl;
or an isomer or salt thereof.

23. A weight loss method comprising administering to a subject in need of weight loss an effective amount of a compound of Formula (III): 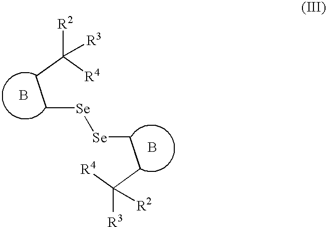
wherein each instance of B is independently an 5-6-membered aryl or heteroaryl group, the heteroaryl group having 1 or 2 heteroatoms selected from the group consisting of N, and S, and wherein the aryl or heteroaryl group may also have a phenyl ring fused thereto and
in each instance B is optionally substituted with 1-3 substituents independently selected from the group consisting of straight or branched —C₁₋₆alkyl, straight or branched —C₂₋₆alkenyl, straight or branched —C₂₋₆alkynyl, —OC₁₋₆, —NO₂, —OH, halo, —CN, and —C₁₋₆(═O)NR^{a3}R^{b3}; wherein in each instance R^{a3} and R^{b3} are independently H or substituted or unsubstituted C₁₋₆alkyl, substituted or unsubstituted —C(═O)C₁₋₆, or substituted or unsubstituted —C(═O)NR^{c3}R^{d3}, wherein one of R^{a3} and R^{b3} is hydrogen, and the other is substituted or unsubstituted phenyl; or wherein R^{a3} and R^{b3} taken together with the nitrogen to which they are attached form a substituted or unsubstituted heterocycloalkyl group;
wherein R^{c3} and R^{d3} are independently H or C₁₋₆alkyl; and
each instance of R² and R³ is hydrogen or —OH, or one or both instances of R² and R³ attached to the same carbon taken together are ═O;
each instance of R⁴ is independently:
i) straight or branched —C₁₋₆alkyl, straight or branched —C₂₋₆alkenyl, straight or branched —C₂₋₆alkynyl, —OC₁₋₆, —C₁₋₃—O—C₁₋₃, —OH, —C₁₋₆NR^{a4}R^{b4}, —C₁₋₆(═S)NR^{a4}R^{b4}, —C₁₋₆(═O)NR^{a4}R^{b4}, —NR^{a4}R^{b4}, —CN, —OCN, —C₁₋₆(═O), —C₁₋₆(═O)OH, or —C₁₋₆(═O)OC₁₋₆, or
ii) cycloalkyl, heterocycloalkyl, aryl, heteroaryl, —C₁₋₆alkylcycloalkyl, —C₁₋₆alkylheterocycloalkyl, —C₁₋₆alkylaryl, or —C₁₋₆alkylheteroaryl (wherein for any ring hetero means 1-2 of N, S, O);
in each instance optionally substituted with 1-4 substituents independently selected from straight or branched —C₁₋₆alkyl, straight or branched —C₂₋₆alkenyl, straight or branched —C₂₋₆alkynyl, —OC₁₋₆, —NO₂, —OH, ═O, halo, —CF₃, —NR^{a4}R^{b4}, —CN, —OCN, —C₁₋₆(═O), —C₁₋₆(═O)OH, —C₁₋₆(═O)OC₁₋₆, —OC₁₋₆(═O)NR^{a4}R^{b4}, —C₁₋₆(═O)NR^{a4}R^{b4}, —C(═O)NHC(═O)C₁₋₆alkyl, —C(═O)NHC(═O)NR^{a4}R^{b4}, —SC₁₋₆alkyl, —SH, —SO₂NR^{a4}R^{b4}, and —N₃;
wherein, in each instance R^{a4} and R^{b4} are independently H or C₁₋₆alkyl, substituted or unsubstituted, or one of R^{a4} and R^{b4} is H and the other is substituted or unsubstituted cycloalkyl, heterocycloalkyl, aryl, heteroaryl, alkylheteroaryl, —C(═O)heterocycloalkyl, —C₁₋₆(═O)OH, —C₁₋₆C(═O)OC₁₋₆, or —C(═N)N—NO₂;
or an isomer or salt thereof.

24. A weight loss method comprising administering to a subject in need of weight loss an effective amount of a compound of Formula (IV): 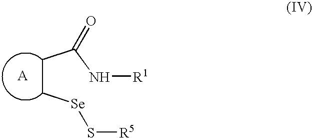
wherein
A is a 5-6-membered aryl or heteroaryl group, which heteroaryl group has 1 or 2 ring atoms selected from the group consisting of N and S;
in each instance optionally substituted with 1-3 substituents independently selected from the group consisting of straight or branched —C₁₋₆alkyl, straight or branched —C₂₋₆alkenyl, straight or branched —C₂₋₆alkynyl, —OC₁₋₆, —NO₂, —OH, halo, —CN, and —CHO;
R¹ is
i) straight or branched —C₁₋₆alkyl, straight or branched —C₁₋₆alkenyl, straight or branched —C₁₋₆alkynyl, —OC₁₋₆, —C₁₋₃—O—C₁₋₃, —OH, —C₁₋₆NR^{a1}R^{b1}, —C₁₋₆(═S)NR^{a1}R^{b1}, —C₁₋₆(O)NR^{a1}R^{b1}, —NR^{a1}R^{b1}, —CN, —OCN, —C₁₋₆(═O), —C₁₋₆(═O)OH, or —C₁₋₆(═O)OC₁₋₆, or
ii) cycloalkyl, heterocycloalkyl, aryl, heteroaryl, —C₁₋₆alkylcycloalkyl, —C₁₋₆alkylheterocycloalkyl, —C₁₋₆alkylaryl, or —C₁₋₆alkylheteroaryl (wherein for any ring hetero means 1-2 of N, S, O);
in each instance optionally substituted with 1-4 substituents independently selected from straight or branched —C₁₋₆alkyl, straight or branched —C₂₋₆alkenyl, straight or branched —C₂₋₆alkynyl, —OC₁₋₆, —NO₂, —OH, ═O, halo, —CF₃, —NR^{a1}R^{b1}, —CN, —OCN, —C₁₋₆(═O), —C₁₋₆(═O)OH, —C₁₋₆(═O)OC₁₋₆, —OC₁₋₆(═O)NR^{a1}R^{b1}, —C₁₋₆(═O)NR^{a1}R^{b1}, —SC₁₋₆alkyl, —SH, —SO₂NR^{a1}R^{b1}, and —N₃; wherein, in each instance R^{a1} and R^{b1} are independently H or substituted or unsubstituted C₁₋₆alkyl, or one of R^{a1} and R^{b1} is H and the other is C₁₋₆ (═O)OH, C₁₋₆C(═O)OC₁₋₆, or —C(═N)N—NO₂, substituted or unsubstituted phenyl or substituted or unsubstituted naphthyl; and
R⁵ is:
i) straight or branched —C₁₋₆alkyl, straight or branched —C₂₋₆alkenyl, straight or branched —C₂₋₆alkynyl, —C₁₋₃—O—C₁₋₃, —C₁₋₆NR^{a5}R^{b5}, —C₁₋₆(═O)NR^{a5}R^{b5}, —NR^{a5}R^{b5}, —C₁₋₆(═O), —C₁₋₆(═O)OH, or —C₁₋₆(═O)OC₁₋₆, or
ii) cycloalkyl, heterocycloalkyl, aryl, heteroaryl, —C₁₋₆alkylcycloalkyl, —C₁₋₆alkylheterocycloalkyl, —C₁₋₆alkylaryl, or —C₁₋₆alkylheteroaryl (wherein for any ring hetero means 1 of N, O);
in each instance optionally substituted with 1-2 substituents independently selected from
straight or branched —C₁₋₆alkyl, halo, —C₁₋₆(═O)OH, and —C₁-6(═O)OC₁₋₆;
wherein, in each instance R^{a5} and R^{b5} are independently H or substituted or unsubstituted C₁₋₆alkyl, or —NR^{a5}R^{b5} is —NH(phenyl), which phenyl is substituted or unsubstituted;
or an isomer or salt thereof.

25. A weight loss method comprising administering to a subject in need of weight loss an effective amount of a compound of Formula (V): 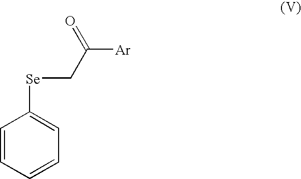
wherein Ar is an aryl or heteroaryl group, which heteroaryl group has 1 ring atom selected from the group consisting of N, S, and O;
in each instance optionally substituted with 1-2 substituents independently selected from the group consisting of straight or branched —C₁₋₆alkyl, —OC₁₋₆, halo, —CN, —OCN, —C₁₋₆(═O), —C₁₋₆(═O)OH, —C₁₋₆(═O)OC₁₋₆, and —OC₁₋₆(═O);
or an isomer or salt thereof.

26. A weight loss method comprising administering to a subject in need of weight loss an effective amount of a compound of Formula (VI): 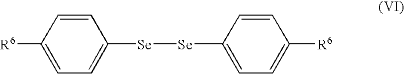
wherein each R⁶ is independently selected from the group consisting of straight or branched —C₁₋₆alkyl, straight or branched —C₂₋₆alkenyl, halo, —CF₃, —NO₂, —CN, —C₁₋₆NR^{a6}R^{b6}, —NR^{a6}R^{b6}, —OH, —OC₁₋₆, —OC₁₋₆(═O) and phenyl,
wherein in each instance R^{a6} and R^{b6} are independently H, substituted or unsubstituted C₁₋₆alkyl; substituted or unsubstituted —C(═O)C₁₋₆, or substituted or unsubstituted —C(═O)NR^{c6}R^{d6}, or wherein one of R^{a6} and R^{b6} is hydrogen, and the other is substituted or unsubstituted phenyl, or wherein R^{a6} and R^{b6} taken together with the nitrogen to which they are attached form a substituted or unsubstituted heterocycloalkyl group and wherein R^{c6} and R^{d6} are independently H or C₁₋₆alkyl;
or an isomer or salt thereof.
